(19) United States
(12) Patent Application Publication
(10) Pub. No.: US 2024/0201151 A1
(43) Pub. Date: Jun. 20, 2024

(54) CALIBRATION GAS SOURCE AND AUTOMATIC CHEMICAL SENSOR CALIBRATION

(71) Applicant: Mission Support and Test Services, LLC, Las Vegas, NV (US)

(72) Inventors: John Di Benedetto, Santa Barbara, CA (US); Ken Moy, Santa Barbara, CA (US)

(21) Appl. No.: 18/595,167

(22) Filed: Mar. 4, 2024

Related U.S. Application Data
(63) Continuation of application No. 17/160,115, filed on Jan. 27, 2021.
(60) Provisional application No. 62/966,467, filed on Jan. 27, 2020.

(51) Int. Cl. G01N 33/00

(52) U.S. Cl. CPC G01N 33/0006

(57) ABSTRACT

A sensor with a built in calibration gas source having an outer housing configured to contain one or more of the sensor elements. Within the housing is a controller configured to manage operation of the sensor and a user interface configured with one or more user input elements and one or more sensor output elements. A detector module has one or more detectors, configured to sense a calibration gas and, responsive to detection of the calibration gas, provide a detection signal to the controller. A calibration module configured to, responsive to a control signal from the controller, generate a calibration gas and present the calibration gas in proximity to one or more of the detectors. A power source is also provided and configured to provide power to the controller, the detector module, and the calibration module.

### 1. STATEMENT REGARDING FEDERAL RIGHTS

This invention mas made with government support under Contract No. DE-NA00036247 effective Dec. 1, 2017, updated to include modifications through 0177, and was awarded by the U.S. Department of Energy, National Nuclear Security Administration. The government has certain rights in the invention.

### 2. FIELD OF THE INVENTION

The invention relates to gaseous chemical sensors and in particular to a self-calibrating gaseous chemical sensor.

### 3. RELATED ART

In many human or animal occupied environments, the possibility exists for chemical contamination. To monitor for and detect chemical contaminates, chemical sensors are placed in human-occupied infrastructure or worn by individuals who may encounter chemical contaminated environments. The chemical sensor systems detect toxic industrial counts (TICs).

Testing and calibration of the sensor occurs to verify accuracy of the sensors over time. Currently, calibration of most chemical sensors used in the field or industrial applications require a “bump” test. This involves passing calibration gas from a cylinder of known chemical contaminate concentration through the portion of the sensor system that contains the detector elements. Typically, during calibration, gases are drawn from a small external chamber fitted to and filled by the gas within the cylinder. This is typically performed by a calibration technician, thus making the process slow, man-power intensive, and costly. For most chemical sensing systems used by first responders and industrial hygiene professionals, sensor systems are calibrated daily. Daily calibration, using the prior art method of calibration, results in the calibration process being a time consuming and expensive drawback to use of chemical sensors.

### SUMMARY

To overcome the drawbacks of the prior art and provide additional benefits, a proposed system and method for automatic calibration functions are disclosed to allow for deployment of chemical sensors over long periods of time where calibration and maintenance are difficult or costly, as well as chemical sensors which can be automatically calibrated in an efficient manner. In addition, the proposed system and method for automatic calibration enables automatic calibration thereby eliminating the need for costly and time-consuming bump tests performed by a calibration technician and cylinder stored chemical contaminated gas.

In one embodiment, the sensor includes a low-power micro ionization source or any other gas source, whether stored or generated, to create an aliquot of gas used to calibrate gas sensor elements inside of a portable detection system. Currently available chemical sensor systems do not have auto-calibration technologies.

In one embodiment, the electron generator is a low voltage, atmospheric pressure field emission array (FEA) located in the systems internal air flow near the detector elements. The FEA is part of a pulsed charging circuit activated intermittently when the system is idle. The circuit creates and discharges a known amount of calibration gas for each pulse and this calibration gas is provided to the detector elements in the sensor to test operation of the detectors. In one embodiment, the FEA generates ozone, however, in other embodiments other calibration gases may be generated. In one embodiment, the FEA will operate at moderately low temperature to prevent thermal decomposition of ozone and may suppress the formation of other gases.

Moderately low temperature is defined herein to mean temperatures at which the generated gas will not decompose before being detected by the detector. In one embodiment the temperature is such that the gas does not break down within a time period sufficient for the detector to detect the gas. This may be 1 second, 3 seconds or 10 seconds, or any other value. The calibration source would be applicable across a large number of detector systems such as but not limited to; metal oxide, photoionization, electrochemical and micro-cantilever. The automatic calibration systems would be useful for industrial, retail and consumer systems where calibration methods are required.

To overcome the drawbacks of the prior art and provide additional benefits, a sensor with a built in calibration gas source is disclosed. In one embodiment, the sensor includes a housing and a controller configured to manage operation of the sensor. Also part of this embodiment, is a user interface, in communication with the controller, that is configured with one or more user input elements that are configured to allow a user to provide input to the sensor, and one or more sensor output elements configured to allow the sensor to provide information to the user. A detector module is provided that has one or more detectors that are configured to sense a calibration gas and, responsive to detection of the calibration gas, provide a detection signal to the controller. A calibration module is configured to, responsive to a control signal from the controller, generate a calibration gas and present the calibration gas in proximity to one or more of the detectors. A power source configured to provide power to the controller, the detector module, and the calibration module.

In one embodiment, the sensor further comprises one or more of a sound generation device, display screen, and visual indicator configured to display results of the self-test to a user. The calibration gas source may be an ionization device. The calibration gas source may be a pressurized container of calibration gas. In one configuration, the calibration gas is ozone. It is contemplated that the power source is a battery or a connection to a wireline power source.

Also disclosed is a method of verifying operation of a sensor comprising initiating a calibration mode of the sensor and activating a calibration gas generator, that is part of the sensor, which causes the ionization source to generate a calibration gas and that is exposed to one or more chemical detectors that are part of the sensor. Responsive to the chemical detector detecting the calibration gas, generating an audio alert, visual alert, or both, indicating detection of the calibration gas.

The step of initiating the calibration mode occurs by a user initiating manually activating calibration mode or automatically at a predetermined time. In one embodiment, this method further comprises, responsive to the sensor not detecting the calibration gas, alerting an audio alert, visual alert, or both indicating that the calibration gas was not detected. It is also contemplated that the calibration gas generator is an ionization source or a field emission array. The calibration gas may be ozone.

Also disclosed is a self-testing chemical sensor comprising a control module configured control activation of test operation and provide a notification responsive to the results of the test operation. Also, part of the sensor is one or more detectors configured to detect at least one calibration gas and, responsive to detecting a calibration gas, provide a notification signal to the control module, and a calibration gas source configured to provide a calibration gas. A power source is part of this sensor and is configured to provide power to one or more of the control module, one or more detectors, and calibration gas source.

In one embodiment, the control module includes a user interface configured to allow a user to manually initiate the self-test mode. This sensor may also have one or more sound generation devices, display screen, and visual indicator configured to display results of the self-test to a user. The calibration gas source may be an ionization source, a field emission array, or a pressurized container of calibration gas. The calibration gas may be ozone, and the power source may be a battery.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a block diagram illustrating an exemplary gaseous chemical generator and sensor system as part of an air path.

FIG. 2 illustrates a block diagram of an example embodiment of a chemical sensor with calibrator including gas source and automatic calibration.

FIG. 3 illustrates an example method of operation of one embodiment of the invention.

### DETAILED DESCRIPTION

Of particular interest are chemical sensors that are placed in human-occupied infrastructure. These chemical sensor systems detect toxic industrial counts (TICs). Currently, calibration of most chemical sensors used in field or industrial applications require a “bump” test. This involves passing calibration gas from a cylinder of known concentration through the portion of the system that contains the detector elements. Typically, gases drawn from a small external chamber fitted to a gas cylinder and provided to the sensor to test the sensor detectors. This is typically completed with a man in the loop. For most chemical sensing systems, used by first responders and industrial hygiene professionals, sensor systems are calibrated daily. Current chemical sensors systems do not have an auto calibration function which prevents the deployment of most chemical sensors over long periods of time where calibration and maintenance are difficult or costly.

This invention provides a calibration gas that can be generated by the sensor itself and release to the sensor elements for periodic calibration. The sensor can be used for calibration or testing, and these terms are used interchangeable herein. The calibration may also include detecting concentrations of the calibration gas in relation to other gases. This helps distinguish the calibration gas from possible background gas and can be used to report to the user the parts per million of the detected gas to verify the detector is accurately detecting the amount of dangerous gas in an environment that may have a tiny, but accepted amount of a dangerous gas.

In one embodiment, ozone can be used as a calibration gas for several sensor types. The calibration could occur weekly, daily, hourly, prior to sensor use, or as often as required for the application. The detector element being calibrated will indicate when there is clear air available and when chemical contaminates are in the sensor environment. The calibration source would be applicable across a large number of detector systems such as, but not limited to; metal oxide, photoionization, electrochemical and micro-cantilever. The device would be useful for military, government, industrial, retail and consumer systems where calibration methods are required or preferred.

In one embodiment, the calibrator uses field emission array (FEA) technology which is typically used in vacuum chambers to make electrons for pulsed power and electroluminescent displays. In this embodiment, a low voltage field emission array (FEA) operates at atmospheric pressure and is placed in the internal air flow upstream or in proximity to the detector elements such that gas generated by the FEA will come in contact with the detector elements.

It is also disclosed that the test or calibration gas may result from piezoelectric direct discharge (PDD) plasma, which is a type of cold non-equilibrium plasma, generated by a direct gas discharge of a high voltage piezoelectric transformer or other similar structure. It can be generated in room temperature air or other gases in a wide range of pressures, including atmospheric pressure. Due to the compactness and the efficiency of the piezoelectric transformer, this method of plasma generation is well suited for the sensor due to this type of plasma generation being compact, efficient and inexpensive to implement. Piezoelectric direct discharge uses a piezoelectric transformer as a generator of AC high voltage. The high voltage side of this transformer acts as an electrode generating electric discharges in the air or other working gases producing atmospheric-pressure plasmas. The piezoelectric transformer is very compact and requires only a source of a low-power low-voltage AC. This allows the whole plasma generator to be made exceptionally compact, enabling construction of hand-held plasma generators or cost-effective plasma generator arrays. The piezoelectric direct discharge generator may generate ozone or other type of gas. One exemplary piezoelectric generator is the CeraPlas Element Piezoelectric Based Cold Plasma Generator, HF Series available from TDK Electronics Ag.

It is also contemplated that the calibration gas may be generated by dielectric barrier discharge, which occurs between two electrodes separated by a dielectric when the electrodes are biased by a sine-wave or pulsed high voltage. The discharge current is sourced from the surface of the dielectric.

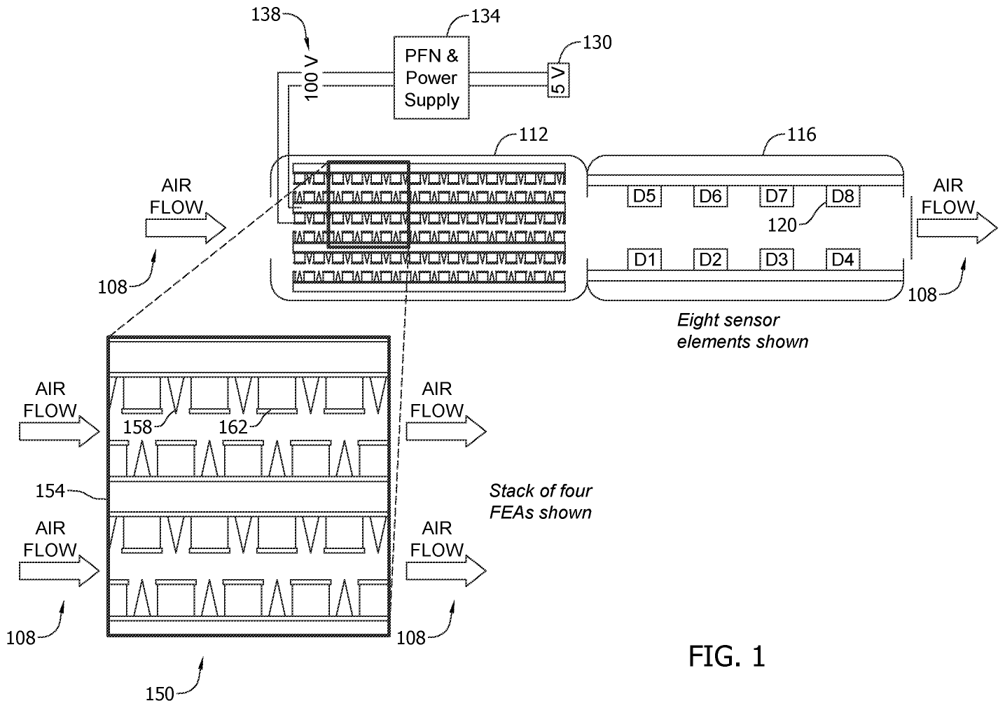

FIG. 1 illustrates one example embodiment of the calibrator technology with an array of detector elements. In this embodiment, metal oxide detectors are shown. The FEA is shown here as part of a pulsed charging circuit that can be energized intermittently (when the system is not making a measurement). The circuit will effectively discharge a known amount of calibration gas for each pulse of electrons. Operation at the FEA's moderately low temperature will help prevent thermal decomposition of ozone and may suppress the formation of other gases. Specific pulse waveforms may be used to optimize the ozone production and help limit the production of some NOX species such as nitric acid. It is also contemplated and disclosed to purposefully generate other gases by this device. The detector may be configured to detect other types of gaseous chemicals and as such, the calibration module having one or more generators may be configured to generate or provide different types of calibration gas to the detector to calibrate (and test) more than one detector type.

Turning now to the embodiment of FIG. 1, an air flow path 108 is provided that passes air through or over a calibration gas generator 112 and a sensor array 116. The sensor array 116 includes one or more sensors 120 which may be configured or tuned to the same gaseous chemical or different gaseous chemicals.

Connecting to the calibration gas generator 112 is a power source. A primary power source 130 may be supplied from a wire connection or a batter, such as high capacity 5 volt battery. The primary power source 130 connects to a PFN and power supply 134. The PFN is a pulse forming network which is an electric circuit that accumulates electrical energy over a comparatively long time, and then releases the stored energy in the form of a pulse of comparatively brief duration for various pulsed power applications. The output from the PFN and power supply 134 is, in this example embodiment, a 100 volt pulse. The 100 volt differential is provided to opposing elements in the calibration gas generator 112.

Enlarged view 150 illustrates an enlarged portion of the calibration gas generator 112. In this example embodiment, the calibration gas generator 112 is a field emission array (FEA). A field emission array (also referred to as a field emitter array) is a particular form of large-area field electron source. FEAs are prepared on a silicon substrate by lithographic techniques similar to those used in the fabrication of integrated circuits. Their structure consists of a very large number of individual, similar, small field electron emitters, usually organized in a regular two-dimensional pattern. A substrate 154 provides a base structure which supports numerous emitter cones 158, also referred to as tips, which is near but not touching numerous gates 162. The emitter cones 158 and gates are at different voltage potentials and the differing voltage potentials resulting from the pulse can generate ozone. The ozone is carried by the air flow 108 to the sensor array for detection.

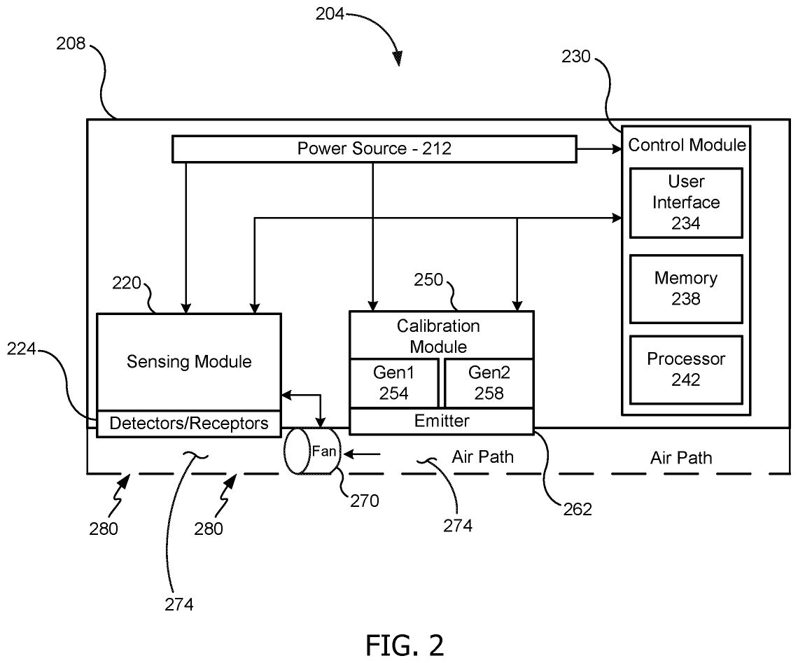

FIG. 2 illustrates a block diagram of an example embodiment of a chemical sensor 204 with calibrator including gas source and automatic calibration. This is but one possible embodiment and other configurations and elements are possible. The arrangement of elements is only exemplary. As shown, a housing 208 encloses and protects the elements on the interior of the housing. The system comprises three general sub-systems. These sub-systems include a sensing system, a calibration system and a control system. A power source 212 connects to each sub-system to provide power, as is understood in the art. The power source 212 may be any power source, such as a wired connection to remote power source, a battery, or any other power source. The power may be supplemented or provided by solar or any other renewable source.

The sensing system includes a sensing module 220 that includes one or more detectors or receptors 224 (hereafter detectors). The sensing module 220 includes one or more circuits that process data from the detector 224 to provide an indicator signal to a control module 230. The detector 224 is configured to generate an electrical signal in response to exposure to a chemical, typically in gas form, to which the detector is tuned. The magnitude or frequency of the indicator signal may be representative of the concentration of the chemical. The sensing module 220 receives and processes the indicator signal from the detector 224 to determine if the detector signal exceeds a threshold, and to generate concentration data. Sensing modules are associated detectors which are known by those of ordinary skill in the art, and as such not described in detail herein. The sensing module 220 communicates with the control module 230. One example of a sensing module is described in U.S. Pat. No. 8,537,020.

The control system includes a control module 230. In this example embodiment the control module 230 includes a user interface 234, a memory 238 and a processor 242. The user interface 234 may comprise one or more of the following: screen, buttons, keyboard, lights, knobs, dials, speakers, buzzers, communication ports, or any other device or system to convey information to a user or receive information from a user. The memory 238 may include volatile or non-volatile memory configured with machine executable instructions, data or both. The machine executable instructions stored on the memory 238 are executable by the processor 242. The processor 242 may comprise any type processing device such as but not limited to a CPU, controller, DSP, ASIC, ARM, control logic, or any combination of these devices or similar devices. The control module 230 oversees operation of the chemical sensor 204 with calibrator. In response to a detection signal from the sensing module 220, the control module provides an alert to the user to provide a warning. The alert may be audible, visual, vibration or any combination thereof.

Also contained within the housings 208 is a calibration module that is part of the calibration system. The term calibration, as used herein, is defined to mean a testing process to verify operation and also determine accuracy of gas or chemical concentration. In this embodiment, the calibration module 250 includes a first generator 254 and a second generator 258. In other embodiments, a single generator may be provided. Connected to each generator 254, 258 is an emitter 262. The calibration module includes control logic to activate the generators 254, 258. The generators 254, 258 provide a calibration gas to the emitter 262. The generators 254, 258 may be any type calibration gas source such as an ozone generator, canister of detectable calibration gas, heat releasable gas source, or any other type of gas source which is detectable by the detector for calibration purposes. A canister or container may be filled with a small amount of pressurized gas which is selectively discharged by the emitter 262. The canister may be periodically replaced by a user when empty to provide a new supply of calibration gas. Further, different gases may be in the container thereby tailoring the sensor and calibration system to various chemical detection when equipped with a heatable chemical emitting material, the material sample emits a chemical embedded therein responsive to heat, electricity or other energy type. Upon heating or otherwise energizing the material, the chemical is emitted, as a gaseous chemical, from the sample into the air path 274 to be used as a calibration gas. The sample may be replaced periodically to restore a supply of the calibration gas after continued use.

The emitter 262 receives the gas from one or more of the generators 254, 258, and provides the calibration gas into an air path 274. The detectors 224 also located in the air path 272 and arranged to receive the emitted calibration gas as part of the calibration processor. Any chemical or gas that is in the environment of the detector 224 and calibration module 250 would also be available to the detector 224 through gaps or vents 280 in the housing which allow environmental air to enter the detector 224 and calibration module 250. An optional fan 270 or other air movement device may be part of this embodiment to direct calibration gas to the detector to ensure that the calibration gas is exposed to the detector 224.

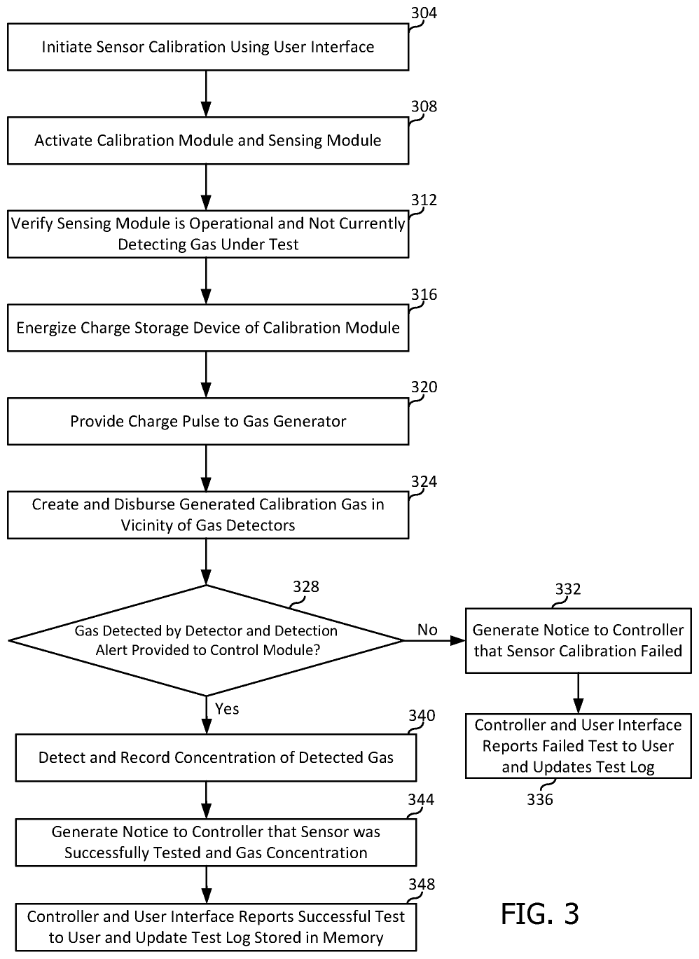

FIG. 3 illustrates an example method of operation. This is but one possible method of operation. At a step 304, a user or the controller within the sensor initiating sensor calibration (or test) using the user interface. The user may initiate the calibration by pushing one or more buttons on the sensor. Alternatively, the sensor may automatically self-calibrate without user input. At a step 308, the sensor controller activates the calibration module 250 and the sensing module 220. To avoid a false calibration test, at a step 312 verification occurs to determine it is operational, such as a built-in self-test, and that the sensor is not currently detecting or exposed to any gas. This verifies status of the detectors.

At a step 316, the controller energizes a charge storage device associated with the calibration module. In some systems, a high voltage pulse may be used to generate the calibration gas. At a step 320, the charge pulse is provided to the gas generator, which causes the gas generator to generate the calibration gas, such as ozone. This may occur one or more times. At a step 324, or as part of step 320, the calibration gas is created and disbursed in the vicinity of the gas detectors. The gas source and detectors may be in close proximity or intermingled. In one embodiment a fan or other air movement device may be used to direct the calibration gas to the sensor.

At a decision step 328, a determination is made if the gas detector has detected the calibration gas. If at decision step 328 the gas detectors have not detected the calibration gas, then the operation advances to step 332, at which the detectors provide a signal or notice to the controller that the calibration gas was not detected, e.g., the calibration test failed. Then, at a step 336, the controller and user interface report the failed calibration test to the user and updates a test log that may be stored in memory. It is contemplated that the sensor may take itself out of service until it is reset and repaired.

Alternatively, if at decision step 328 the calibration gas was detected by the detector, then the operation advances to step 340. At step 340, the detector records the concentration of the detected calibration gas. Then at a step 344, the detector generates and send a notice or signal to the controller that the detector successfully detected the calibration gas and gas concentration. Responsive thereto, at a step 348, the controller and user interface report the successful calibration to the user and updates the test log stored in memory.

Additional or fewer components may be utilized to enable the gaseous chemical detector with automatic calibration. While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A sensor with a built in calibration gas source comprising:
a user interface configured with one or more user input elements configured to receive input from a user and one or more sensor output elements configured to provide information to the user;
a sensor module, having one or more gas detectors, configured to sense a calibration gas and, responsive to detection of the calibration gas, provide a detection signal to the controller;
a calibration module comprising a field emission array or a piezoelectric direct discharge transformer which function as a calibration gas generator, configured to, responsive to a control signal from the controller, generate a calibration gas within the sensor and present the calibration gas in proximity to one or more of the detectors;
a power source configured to provide power to the controller, the sensor module, and the calibration module;
a controller, having a user interface, the controller configured to manage operation of the sensor, the controller configured to:
receive input from a user via the user input elements of the user interface to initiate a sensor self-test mode;
responsive to the input from the user, initiate the sensor self-test mode and send a signal to the calibration module to initiate generation of the calibration gas;
responsive to the sensor module detecting the calibration gas, providing a notice on at least one of the one or more sensor output elements of the detection of the calibration gas that the calibration gas was detected and the sensor is functioning; and
a housing enclosing the controller, user interface, sensor module, calibration module, and power source.

2. The sensor of claim 1 wherein the sensor output elements comprise one or more of a sound generation device, display screen, and visual indicator, which are part of the sensor, and connect to and receive input from the controller, and are configured to alert the user if the sensor module detected the calibration gas.

3. The sensor of claim 1 wherein the calibration gas generator is an ionization device.

4. The sensor of claim 1 further comprising a fan configured to generate air movement which carries the calibration gas to sensor module.

5. The sensor of claim 1 wherein the calibration gas is ozone.

6. The sensor of claim 1 wherein the power source is a battery or a connection to a wireline power source.

7. A method of verifying operation of a sensor comprising:
receiving user input to initiate a self-test of the sensor;
responsive to the user input, initiating a test mode of the sensor to perform the self-test;
activating a test gas generator that comprises a field emission array or a piezoelectric direct discharge transformer, that is part of and contained within the sensor, which generates a test gas within the sensor;
exposing one or more chemical detectors to the test gas, the one or more chemical detectors are part of and contained within the sensor;
monitoring for the test gas with the one or more chemical detectors; and
responsive to the one or more chemical detectors detecting the test gas, generating an audio alert, visual alert, or both indicating detection of the test gas thereby verifying operation of the sensor.

8. The method of claim 7 wherein verifying operation of the sensor occurs automatically at a predetermined time.

9. The method of claim 7 further comprising, responsive to the sensor not detecting the test gas, providing an audio alert, visual alert, or both indicating that the test gas was not detected.

10. The method of claim 7 wherein the test gas generator is an ozone source.

11. The method of claim 7 wherein the test gas generator is a field emission array having two or more emitter cones and two or more gates.

12. The method of claim 7 wherein the test gas is ozone.

13. A self-testing chemical sensor comprising:
a control module in communication with one or more detectors and a calibration gas source configured control activation of a test operation of the chemical sensor and provide a notification responsive to the results of the test operation, wherein the control module comprises a memory, a processor, and a user interface;
the one or more detectors configured to detect at least one calibration gas and, responsive to detecting at least one calibration gas, provide a detection notification signal to the control module;
the calibration gas source comprising a field emission array or a piezoelectric direct discharge transformer within the sensor that is configured to generate and provide the at least one calibration gas to the one or more detectors responsive to activation of the test operation by the control module; and
a power source configured to provide power to one or more of the control module, one or more detectors, and calibration gas source.

14. The self-testing chemical sensor of claim 13 wherein the control module includes a user interface configured to allow a user to manually initiate the self-test mode.

15. The self-testing chemical sensor of claim 13 further comprising one or more of a sound generation device, display screen, and visual indicator which connect to and receive input from the control module, and responsive to the input from the control module, display results of the test operation to a user.

16. The self-testing chemical sensor of claim 13 wherein the calibration gas source is an ionization source.

17. The self-testing chemical sensor of claim 13 wherein the calibration gas source is a field emission array having two or more emitter cones and two or more gates.

18. The self-testing chemical sensor of claim 13 wherein the calibration gas source is a pressurized container of calibration gas contained within the sensor.

19. The self-testing chemical sensor of claim 13 wherein the calibration gas is ozone.

20. The self-testing chemical sensor of claim 13 wherein the power source is a battery.
